(19) United States
(12) Patent Application Publication
(10) Pub. No.: US 2010/0055682 A1
(43) Pub. Date: Mar. 4, 2010

(54) Procedure and methods for detecting alzheimers's disease

(76) Inventors: Fabien Schweighoffer, Nogent sur Marne (FR); Laurent Bracco, La Garenne Colombes (FR)

Correspondence Address: NIXON & VANDERHYE, PC, 901 NORTH GLEBE ROAD, 11TH FLOOR, ARLINGTON, VA, 22203, US

(21) Appl. No.: 12/227,374

(22) PCT Filed: May 14, 2007
(86) PCT No.: PCT/FR2007/051263

§ 371 Date: Feb. 11, 2009

(30) Foreign Application Priority Data
May 15, 2006 (FR) 0604294

(51) Int. Cl. C12Q 1/68; C07H 21/02; C07D 219/08; C07D 211/06; C07C 271/44

(52) U.S. Cl. 435/6; 536/23.1; 546/105; 546/206; 560/163

(57) ABSTRACT

This application concerns methods and compositions that can be used for detecting the Alzheimer disease in mammals, particularly in humans. It describes in particular serum markers for Alzheimer's disease and the way they are used for diagnostic procedures. It also concerns tools and/or kits that can be used for applying these procedures (reagents, probes, primers, antibodies, chips, cells, etc.) with the preparation thereof and the way to use them. The invention can be used to detect the presence or the progression of Alzheimer illness in mammals, including at early stages of the disease.

The present application relates to methods and compositions that can be used to detect Alzheimer's disease in mammals, in particular in humans. It describes in particular serum markers for Alzheimer's disease and uses of same in diagnostic procedures. It also relates to tools and/or kits that can be used to apply these methods (reagents, probes, primers, antibodies, chips, cells, etc.), and preparation and use of same. The invention can be used to detect the presence or the progression of Alzheimer's disease in mammals, including at early stages of the disease.

Alzheimer's disease is the principal cause of dementia and the most common neurodegenerative disease. This disease, progressive in nature, is characterized by memory loss and degradation of language skills, orientation and judgment. The nature of the symptoms, which are often confused with the physiological troubles of old age, their severity and the age at which they appear vary from individual to individual. This contributes to the difficulty in establishing a diagnosis during the early stages of the disease.

Examination of the brains of patients suffering from this disease reveals a loss of neurons in the hippocampus, an important center for memory, and in the cerebral cortex, which is involved in reasoning, language and memory. Cholinergic neurons are particularly affected by this depletion.

Another major anomaly observed in the brains of patients suffering from Alzheimer's disease is the accumulation of intracellular and extracellular protein aggregates. Intracellular neurofibrillary tau protein tangles appear well correlated with degree of dementia. Senile plaques formed by the intracellular and extracellular aggregation of amyloid-beta peptide characterize modified regions of neurons and glial cells.

It is however remarkable to note that these regions of aggregation do not correspond to the sites of synaptic depletion characteristic of the decline in cognitive functions.

Genetic studies undertaken on the hereditary forms have shown that four genes are associated with the development of the disease: APP (amyloid precursor protein), presenilins 1 and 2 (PS1 and PS2) and apolipoprotein E (ApoE). Although mutations or polymorphisms in each of these genes lead to increased production of amyloid-beta peptide, the mechanisms that govern synaptic and neuronal losses remain poorly understood. In this respect, several hypotheses and mechanisms involving various phenomena thus appear to coexist:

1) Purely cerebral phenomena involving neurons and glial cells:
oxidative stress, which can be induced in particular by the amyloid-beta peptide and modulated by cholesterol metabolism;
changes in calcium flux and excitotoxicity;

2) Inflammatory and immune reaction phenomena;

3) Changes in sex hormones;

4) Hypothyroidism and defects in insulin signal regulation.

Consequently, Alzheimer's disease is characterized by a change in the various integrated systems that regulate homeostasis, with the attack on certain neurons leading both to an inflammatory reaction involving the immune system and to modifications of endocrine regulation. The latter have in return an impact on the activity and viability of other neurons and on immune functions, these cascading reactions underlining the role not only of neurodegeneration but also of hormone regulation and the immune response in the progression of Alzheimer's disease.

Currently there is no robust and specific signature for Alzheimer's disease that makes it possible to diagnose this pathology, most notably the various stages of the progression of the disease. The availability of an effective diagnostic test, in particular an early test, would enable patients to be cared for at the onset of the disease and thus to benefit from a more effective and more suitable treatment, most notably by acetylcholinesterase inhibitors such as tacrine, donepezil and rivastigmine under optimal conditions.

The present invention responds to this need. The invention describes in particular the identification of serum markers for Alzheimer's disease, allowing the development of effective and predictive diagnoses of the presence of, the stage of or the risk of developing this disease. The invention thus describes the identification of molecular signatures specifically or preferentially expressed in the blood of patients suffering from Alzheimer's disease, resulting in particular from alternative splicing. In a particularly unexpected way, the present application demonstrates the existence, in the blood cells of patients suffering from Alzheimer's disease, of a dysregulation of the internal clock and of molecular signaling pathways involved in phagocytosis and/or oxidative stress. The invention can thus provide, for the first time, tools and methods for diagnosing, predicting and/or monitoring the progression of Alzheimer's disease, based on measuring, in the blood of subjects, the expression of one or more genes chosen among the genes regulated according to a circadian rhythm and the genes involved in regulating phagocytosis or oxidative stress. The presence of dysregulation in the expression of such genes makes it possible to establish the risk of or the predisposition to Alzheimer's disease, or to confirm the presence of this pathology in a subject.

One object of the invention thus resides in a method for detecting (in vitro or ex vivo) the presence of or the risk of developing Alzheimer's disease in a mammal, comprising the determination of the presence, in a biological sample from the mammal, preferably in a (derivative) blood sample, of a change in one or more genes or RNA chosen among the genes regulated according to a circadian rhythm and the genes involved in regulating phagocytosis or oxidative stress, the presence of such a change being indicative of the presence of or the risk of developing Alzheimer's disease in said mammal.

Another object of the invention relates to a method for evaluating or monitoring the effectiveness of a treatment for Alzheimer's disease, comprising a step of measuring the expression of one gene or, preferably, several genes chosen among the genes regulated according to a circadian rhythm and the genes involved in regulating phagocytosis or oxidative stress, during treatment, and comparing the expression thus measured with that measured at a previous stage of treatment.

Another object of the invention relates to improved methods for treating Alzheimer's disease, the improvement comprising of measuring the expression of one gene or, preferably, several genes chosen among the genes regulated according to a circadian rhythm and the genes involved in regulating phagocytosis or oxidative stress, in a subject, before and/or during treatment. Measuring such expression makes it possible to adapt the treatment according to the progression of the pathology. The treatment is typically one using acetylcholinesterase inhibitors such as tacrine, donepezil and rivastigmine.

Another object of the invention relates to the use of an acetylcholinesterase inhibitor such as tacrine, donepezil and rivastigmine to prepare a drug to treat Alzheimer's disease in a patient with a dysregulation of the expression of (at least) one gene as defined above.

In the context of the invention, change in a gene or in RNA means (i) any change in expression, namely dysregulation of levels of expression (e.g., transcription or translation); dysregulation of splicing, leading for example to the appearance of particular spliced forms or to a change in the quantity (relative) of or the ratio between the various splicing forms; as well as (ii) any change in the structure of the protein produced (appearance or disappearance of truncated, elongated or mutated forms, etc.).

As will be described in the text that follows, the present application describes the identification of dysregulation in the splicing of molecules that participate in the signal cascades involved in the circadian rhythm and in the regulation of phagocytosis or oxidative stress in the blood of patients suffering from Alzheimer's disease. Any molecule or technique that measures the expression of these genes in the blood, such as nucleotide primers, nucleotide probes or specific antibodies, which can be in suspension or in immobilized form, can be implemented within the framework of the present invention as will be described in detail in the text that follows.

Thus, another object of the present application relates to a product comprising a substrate on which are immobilized nucleic acids comprising a sequence complementary to and/or specific for one gene or, preferably, several genes or RNAs such as defined above. Preferably, the product comprises distinct nucleic acids comprising a complementary to and/or specific sequence for at least 5, 10, 20, 30, 40, 50, 60 or more genes or RNAs such as defined above.

Another object of the present application relates to a product comprising a substrate on which is immobilized at least one ligand of a polypeptide coded by a gene or RNA such as defined above. Preferably, the product comprises at least 5, 10, 20, 30, 40, 50, 60 or more ligands of different polypeptides chosen among the polypeptides mentioned above.

Another object of the present application relates to a kit comprising a compartment or container comprising at least one nucleic acid, preferably several nucleic acids, comprising a complementary and/or specific sequence for one or more genes or RNAs such as defined above and/or one ligand, preferably several ligands, of one or more polypeptides such as defined above. Preferably, the product comprises at least 5, 10, 20, 30, 40, 50, 60 or more different nucleic acids and/or ligands chosen among the nucleic acids and ligands mentioned above. The kit can also comprise reagents for a hybridization or immunological reaction, as well as, if need be, controls and/or instructions.

Another object of the invention relates to the use of a product or kit such as defined above for detecting Alzheimer's disease in a mammalian subject, preferably a human subject.

Serum Markers for Alzheimer's Disease

The present invention rests on revealing and characterizing serum biological events characteristic of Alzheimer's disease in a human patient. These events constitute biomarkers whose detection in a patient makes it possible, preferably in combination, to determine, even at an early stage, the risk of developing such a disease, the presence of such a disease, or the stage of progression of this disease. Moreover, the inventive markers can also be used to measure the effectiveness of a treatment, and/or to select candidate drugs. Combinations of the inventive markers can make it possible to distinguish Alzheimer's disease from other neurodegenerative pathologies.

The identified biological events typically correspond to modifications in the regulation of gene expression. It can be a question of partial or total inhibition of the expression of genes or RNAs, or of certain forms of genes or RNAs, an increase in the expression of genes or of certain forms of genes or RNAs, the appearance or disappearance of splicing forms of genes, etc.

In the context of the invention, the phrase “gene regulated according to a circadian rhythm” means any gene whose expression is regulated by the internal biological clock. It relates in particular to any RNA or any protein whose expression or activity is regulated according to a chronological rhythm, for example a periodicity of 24 h. In man, the circadian rhythm is controlled by a “biological clock” (the suprachiasmatic nucleus) located in the hypothalamus. It in turn controls other biological clocks, such as those controlling the thermal rhythm of the body or the synthesis of hormones. As examples illustrating genes regulated according to a circadian rhythm for the implementation of the invention, all of the genes mentioned in table 1 can be cited in particular.

Thus, a particular object of the invention resides in a method for detecting (in vitro or ex vivo) the presence of or the risk of developing Alzheimer's disease in a mammal, comprising the determination of the presence, in a biological sample from the mammal, preferably in a sample (derivative) of blood, of a change in one or more genes indicated in table 1, or in the corresponding RNA, in particular of a change in the splicing of one such gene or RNA, with the presence of such a change being indicative of the presence of or the risk of developing Alzheimer's disease in this mammal.

In a particular embodiment, the inventive method comprises at least the determination of the presence, in a biological sample from a mammal, preferably in a sample (derivative) of blood, of a change in the BMAL1 or CLOCK gene, or in a corresponding RNA, in particular of a change in the splicing of one such gene or RNA.

In the context of the invention, the phrase “gene involved in regulating phagocytosis or oxidative stress” means any gene whose expression product takes part in the mechanism of phagocytosis or oxidative stress in blood cells. Phagocytosis is the mechanism by which certain living cells engulf and digest certain foreign particles. As examples illustrating such genes in the context of the invention, all of the genes mentioned in table 2 can be cited in particular.

Thus, another particular object of the invention resides in a method for detecting (in vitro or ex vivo) the presence of or the risk of developing Alzheimer's disease in a mammal, comprising the determination of the presence, in a biological sample from the mammal, preferably in a sample (derivative) of blood, of a change in one or more genes indicated in table 2, or in the corresponding RNA, in particular of a change in the splicing of one such gene or RNA, with the presence of such a change being indicative of the presence of or the risk of developing Alzheimer's disease in this mammal.

In a particular embodiment, the inventive method comprises at least the determination of the presence, in a biological sample from a mammal, preferably in a sample (derivative) of blood, of a change in the L-plastin or calnexin gene, or in a corresponding RNA, in particular of a change in the splicing of one such gene or RNA.

Preferably, the invention rests on the combined detection of a change in several genes chosen among the genes mentioned above. The term “combined detection” means that the change in several genes is determined in order to give rise to an evaluation, this determination being performed simultaneously or non-simultaneously. Thus, a particular embodiment of the invention involves the combined detection of a change in at least one gene regulated by the circadian rhythm and in at least one gene involved in regulating phagocytosis or oxidative stress.

The invention thus rests on detecting, in a sample, one or more target molecules advantageously selected among:

a) the genes indicated in tables 1 and 2, or the corresponding RNAs, or a distinctive fragment of same having at least 15, preferably at least 16, 17, 18, 19, 20, 25 or 30 consecutive bases,

b) the nucleic acids having a sequence complementary to a sequence according to a),

c) the functional analogues of the nucleic acids according to a) or b), or

d) the polypeptides coded by the nucleic acids according to a) to c).

The target molecule can be the complete sequence of the gene or RNA or corresponding protein, or a distinctive fragment of same, i.e., a fragment whose sequence is specific to said gene or RNA, or to said protein, and/or which comprises a variable domain (splicing, deletion, polymorphism, etc.) representative of the biological event to detect.

The term “functional analogue” means an analogue from another mammalian species. Indeed, the genes listed in tables 1 and 2 are human genes, and these sequences constitute markers that are effective and suited for detecting Alzheimer's disease in human patients. Nevertheless, to apply the inventive methods to other mammalian species it is generally preferable to use functional analogues of these sequences, characterized in the species considered. These analogues can be identified by any technique known to those persons skilled in the art, notably in consideration of the sequences provided in the application and the names of the corresponding genes.

In a particular embodiment, the method comprises the determination of the presence of at least one nucleic acid according to a) to c).

In a quite particular embodiment, the method is used to detect Alzheimer's disease in a human subject and comprises the determination of the presence of at least one nucleic acid according to a) or b).

Methods of Detecting a Change in a Gene

As indicated previously, a change in a gene or RNA means in the context of the invention (i) any change in expression, namely dysregulation of levels of expression (e.g., transcription or translation); dysregulation of splicing, leading for example to the appearance of particular spliced forms or to a change in the quantity (relative) of or the ratio between the various splicing forms; as well as (ii) any change in the structure of the protein produced (appearance or disappearance of truncated, elongated or mutated forms, etc.).

Various techniques that enable the detection of a species of nucleic acid in a sample can be used in the present invention, such as for example Northern blot, selective hybridization, the use of substrates covered with oligonucleotide probes, amplification of nucleic acids such as for example by RT-PCR, quantitative PCR or ligation-PCR, etc. These methods can comprise the use of a nucleic probe (for example an oligonucleotide) able to detect selectively or specifically the nucleic acid targets in the sample. Amplification can be carried out according to various methods known to those persons skilled in the art, such as PCR, LCR, transcription mediated amplification (TMA), strand displacement amplification (SDA), NASBA, the use of allele specific oligonucleotides (ASO), allele specific amplification, Southern blot, single-strand conformation analysis (SSCA), in situ hybridization (e.g., FISH), gel migration, heteroduplex analysis, etc. If necessary, the quantity of nucleic acid detected can be compared with a reference value, for example a median or mean value observed among patients not suffering from Alzheimer's disease, or with a value measured in parallel in a control sample. Thus, it is possible to reveal a variation in levels of expression.

According to a preferred embodiment, the method comprises the detection of the presence, absence or quantity (relative) of a nucleic acid according to a) to c) by selective hybridization or selective amplification.

Selective hybridization is typically carried out using nucleic probes, preferably immobilized on a substrate, such as a solid or semi-solid substrate with at least one surface, flat or not, allowing the immobilization of nucleic probes. Such substrates include, for example, strips, beads, membranes, filters, columns, plates, etc. They can be made of any compatible material, notably such as glass, silica, plastic, fiber, metal, polymer, etc. Nucleic probes can be any nucleic acid (DNA, RNA, PNA, etc.), preferably single strand, comprising a sequence specific to a target molecule such as defined in a) to c) above. Probes typically comprise from 5 to 400 bases, preferably from 8 to 200, more preferentially less than 100, and even more preferentially less than 75, 60, 50, 40 or even 30 bases. Probes can be synthetic oligonucleotides, produced on the basis of sequences of target molecules of the invention according to classical synthesis techniques. Such oligonucleotides typically comprise from 10 to 50 bases, preferably from 20 to 40, for example approximately 25 bases. In a particularly advantageous embodiment, several different oligonucleotides (or probes) are used to detect the same target molecule. It can be a question of oligonucleotides specific for different regions of the same target molecule, or centered differently on the same region. Also of use are pairs of probes, one of which is perfectly matched with the target molecule while the other is mismatched, thus allowing an estimation of background noise. The probes can be designed to hybridize with a region of an exon or an intron, or with a region comprising an exon-exon, exon-intron or intron-intron junction. Thus, the probes make it possible to reveal and to distinguish various splicing forms of a gene.

The probes can be synthesized beforehand and then deposited on the substrate, or synthesized directly on the substrate in situ according to methods known to those persons skilled in the art. The probes can also be produced by genetic techniques, for example by amplification, recombination, ligation, etc.

The probes thus defined constitute another object of the present application, as well as the use of same (primarily in vitro) for detecting Alzheimer's disease in a subject.

Hybridization can be carried out under classical conditions known to and adjusted by those persons skilled in the art (Sambrook, Fritsch, Maniatis (1989) Molecular Cloning, Cold Spring Harbor Laboratory Press). In particular, hybridization can be carried out under conditions of high, medium or low stringency, according to the level of sensitivity sought, the quantity of material available, etc. For example, suitable hybridization conditions include a temperature between 55° C. and 63° C. for 2 hours to 18 hours. Other hybridization conditions, adapted to high density substrates, are for example a hybridization temperature between 45° C. and 55° C. After hybridization, various washings can be carried out to eliminate the non-hybridized molecules, typically in SSC buffers comprising SDS, such as a buffer comprising 0.1× to 10×SSC and 0.5-0.01% SDS. Other washing buffers containing SSPE, MES, NaCl or EDTA can also be used.

In a typical implementation, the nucleic acids (or chips or substrates) are pre-hybridized in a hybridization buffer (Rapid Hybrid Buffer, Amersham) typically containing 100 μg/ml of salmon sperm DNA at 65° C. for 30 min. The sample nucleic acids are then placed in contact with the probes (typically applied to the substrate or the chip) at 65° C. for 2 hours to 18 hours. Preferably, the sample nucleic acids are labeled beforehand by any known label (radioactive, enzymatic, fluorescent, luminescent, etc.). The substrates are then washed in a buffer of 5×SSC with 0.1% SDS at 65° C. for 30 min, and then in a buffer of 0.2×SSC with 0.1% SDS. The hybridization profile is analyzed according to classical techniques, such as for example by measuring labeling on the substrate by means of a suitable instrument (for example InstantImager, Packard Instruments). Hybridization conditions can naturally be adjusted by those persons skilled in the art, for example by modifying the hybridization temperature and/or the salt concentration of the buffer as well as by adding auxiliary substances such as formamide or single-strand DNA.

A particular object of the invention thus resides in a method for detecting the presence of or the risk of developing Alzheimer's disease in a mammal, or for evaluating the effectiveness of a treatment for Alzheimer's disease, comprising the placing in contact, under conditions enabling hybridization between complementary sequences, of the nucleic acids from a mammalian blood sample and from a specific set of probes for the target molecules identified previously to obtain a hybridization profile, the hybridization profile being characteristic of the presence of or the risk of developing Alzheimer's disease in this mammal, or of the effectiveness of treatment.

Selective amplification is preferably carried out using a primer or a pair of primers enabling amplification of all or part of one of the nucleic acid targets in the sample, when one such target is present. The primer can be specific for a target sequence such as defined above, or for an area flanking the target sequence in a nucleic acid of the sample. The primer typically comprises a single-strand nucleic acid of a length advantageously between 5 and 50 bases, preferably between 5 and 30 bases. Such a primer constitutes another object of the present application, as well as the use of same (primarily in vitro) for detecting Alzheimer's disease in a subject. The primers can be designed to hybridize with a region of an exon or an intron, or with a region comprising an exon-exon, exon-intron or intron-intron junction. Thus, the primers make it possible to reveal and to distinguish various splicing forms of a gene.

In this respect, another object of the invention resides in the use of a nucleotide primer or a set of nucleotide primers enabling the amplification of all or part of one gene, preferably several genes, mentioned in tables 1 and 2, or the corresponding RNAs, to detect the presence of or the risk of developing Alzheimer's disease in a mammal, or to evaluate the effectiveness of a treatment for Alzheimer's disease in a mammal, particularly in a human being.

Detecting a Change in a Polypeptide

In another embodiment, the method comprises the determination of the presence or the quantity (relative) of a polypeptide coded by a gene such as defined previously. Revealing or assaying a polypeptide in a sample can be carried out by any technique known to those persons skilled in the art, such as in particular by means of a specific ligand, for example an antibody or a fragment or an antibody derivative. Preferably, the ligand is a specific antibody of the polypeptide, or a fragment of one such antibody (for example Fab, Fab′, CDR, etc.), or a derivative of one such antibody (for example a single-chain antibody, ScFv). The ligand is typically immobilized on a substrate, such as a strip, bead, column, plate, etc. The presence or the quantity of the target polypeptide in the sample can be detected by revealing a complex between the target and the ligand, for example by using a labeled ligand, by using a second labeled detection ligand, etc. Immunological techniques that can be used and are well known include ELISA, RIA, etc. If necessary, the quantity of polypeptide detected can be compared with a reference value, for example a median or mean value observed among patients not suffering from Alzheimer's disease, or with a value measured in parallel in a control sample. Thus, it is possible to reveal a variation in levels of expression.

Specific antibodies of target polypeptides can be produced by conventional techniques, in particular by immunizing a non-human animal with an immunogen comprising the polypeptide (or an immunogenic fragment of same) and recovering the antibodies (polyclonal) or the producing cells (to produce monoclonal antibodies). Techniques for producing polyclonal or monoclonal antibodies, ScFv fragments and human or humanized antibodies are described for example in Harlow et al., Antibodies: A Laboratory Manual, CSH Press, 1988; Ward et al., Nature 341 (1989) 544; Bird et al., Science 242 (1988) 423; WO94/02602; U.S. Pat. No. 5,223,409; U.S. Pat. No. 5,877,293; WO93/01288. The immunogen can be produced by synthesis, or by expression, in a suitable host, of a nucleic acid target such as defined above. Such an antibody, monoclonal or polyclonal, as well as its derivatives having the same antigenic specificity, also constitutes an object of the present application, as well as the use of same to detect cancer.

Modifications of the expression and/or structure of proteins can also be detected by means of techniques known to those persons skilled in the art involving mass spectroscopy, more generally grouped under the name proteomic analysis, in order to detect specific signatures in the blood of patients suffering from Alzheimer's disease.

Implementation of the Method

The inventive method is applicable to any biological sample from the mammal tested, in particular any sample comprising nucleic acids or polypeptides. Advantageously, a sample of blood, plasma, platelet, saliva, urine, stool, etc., can be cited, more generally any tissue, organ or, advantageously, biological fluid comprising nucleic acids or polypeptides.

In a preferred and particularly advantageous embodiment, the sample is a sample derived from blood, for example a sample of blood, serum or plasma. The invention follows indeed from the identification of blood markers for Alzheimer's disease, and thus enables detection of this pathology without a tissue biopsy, but only from blood samples.

The sample can be obtained by any known technique, for example by drawing blood, by noninvasive techniques, from sample collections or banks, etc. The sample can also be pretreated to facilitate the accessibility of the target molecules, for example by lysis (mechanical, chemical, enzymatic, etc.), purification, centrifugation, separation, etc. The sample can also be labeled to facilitate the determination of the presence of the target molecules (fluorescent, radioactive, luminescent, chemical, enzymatic labeling, etc.).

In a preferred embodiment, the biological sample is a sample of whole blood, i.e., one not having undergone a step of separation, and optionally can be diluted.

Preferably, the method comprises the combined determination of the presence, absence or quantity of 5, 10, 20, 30, 40, 50 or 60 target molecules such as defined above.

Another particular object of the present application relates to a method for detecting the presence of, the progression of or the risk of developing Alzheimer's disease in a human subject, comprising the placing in contact of a biological sample from the subject containing nucleic acids with a product comprising a substrate on which are immobilized nucleic acids comprising a complementary and/or specific sequence for one gene or, preferably, several genes or RNAs such as defined above and determination of the hybridization profile, the profile indicating the presence of, the stage of or the risk of developing Alzheimer's disease in said human subject. Preferably, the product comprises distinct nucleic acids comprising a complementary and/or specific sequence for at least 5, 10, 20, 30, 40, 50, 60 or more different genes or RNAs as mentioned above.

Another object of the present application relates to a product comprising a substrate on which are immobilized nucleic acids comprising a complementary and/or specific sequence for one gene or, preferably, several genes or RNAs such as defined above. Preferably, the product comprises distinct nucleic acids comprising a complementary and/or specific sequence for at least 5, 10, 20, 30, 40, 50, 60 or more genes or RNAs such as defined above.

Another object of the present application relates to a product comprising a substrate on which at least one ligand of a polypeptide coded by a gene or RNA such as defined above is immobilized. Preferably, the product comprises at least 5, 10, 20, 30, 40, 50, 60 or more ligands of different polypeptides chosen among the polypeptides mentioned above.

The substrate can be any solid or semi-solid substrate having at least one surface, flat or not (i.e., in two or three dimensions), enabling the immobilization of nucleic acids or polypeptides. Such substrates include for example strips, beads, membranes, filters, columns, plates, etc. They can be made of any suitable material, such as in particular glass, silica, plastic, fiber, metal, polymer, polystyrene, Teflon, etc. The reagents can be immobilized on the surface of the substrate by known techniques, or, in the case of nucleic acids, synthesized directly on the substrate in situ. Immobilization techniques include passive adsorption (Inouye et al., J. Clin. Microbiol. 28 (1990) 1469) and covalent bonding. Techniques are described for example in WO90/03382 and WO99/46403. The reagents immobilized on the substrate can be arranged according to a pre-established scheme to facilitate detection and identification of the complexes formed, according to a variable and adaptable density.

In one embodiment, the inventive product comprises multiple synthetic oligonucleotides, between 5 and 100 bases in length, specific for one or more genes or RNAs such as defined above.

The inventive products typically comprise control molecules that make it possible to calibrate and/or standardize the results.

Another object of the present application relates to a kit comprising a compartment or container comprising at least one nucleic acid, preferably several nucleic acids, comprising a complementary and/or specific sequence for one or more genes or RNAs such as defined above and/or one ligand, preferably several ligands, of one or more polypeptides such as defined above. Preferably, the product comprises at least 5, 10, 20, 30, 40, 50, 60 or more different nucleic acids and/or ligands chosen among the nucleic acids and ligands mentioned above. The kit can also comprise reagents for a hybridization or immunological reaction, as well as, if need be, controls and/or instructions.

Another object of the invention relates to the use of a product or kit such as defined above for detecting Alzheimer's disease in a mammalian subject, preferably a human subject.

Other aspects and advantages of the present invention will appear upon consideration of the following examples, which should be regarded as illustrative and nonrestrictive.

### EXAMPLE 1

### Identification of Serum Markers for Alzheimer's Disease Related to the Circadian Rhythm

A genetic analysis was carried out using RNA extracted from the blood of patients suffering from Alzheimer's disease with various degrees of progression of the disease, on the one hand, and using RNA prepared from the blood of healthy control subjects with an mean age identical to that of the sick subjects, on the other hand.

The signatures thus obtained were analyzed by bioinformatic techniques and made it possible to identify the molecular basis of dysregulation phenomena in Alzheimer's disease.

Thus was identified a signature that derives from the mRNA coding for BMAL1 and that corresponds to nucleotides 128 to 247 of GenBank sequence AB000816. BMAL1, as does the CLOCK gene, codes for a transcription factor of the family of basic-helix-loop-helix (bHLH)—PAS domain containing transcription factors. The products of BMAL1 and CLOCK form a transcription complex involved in regulating the internal clock and regulating circadian rhythms. Consequently, the present invention describes, for the first time, a dysregulation of the circadian rhythm of blood cells of patients suffering from Alzheimer's disease. The circadian system in mammals involves central and peripheral oscillators. A hierarchical predominance has been shown between the circadian system of the central nervous system and that of the peripheral tissues. Thus, the central system ensures peripheral synchronization. The region of the brain that controls this central internal clock is changed during Alzheimer's disease and the present invention documents for the first time a repercussion on the level of the blood cells of a dysregulation of the internal clock. Table 1 below gives a list of the genes controlled according to the circadian rhythm, which constitute targets in the context of the present invention.

TABLE 1

| | | | |
|---|---|---|---|
| | BMAL1 | | AB000816 |
| | CLOCK | | |
| 00007 | Arylalkylamine N- | 17q25 | NM_001088 |
| | acetyltransferase | | |
| 00009 | Aryl hydrocarbon receptor nuclear | 11p15 | NM_001178 |
| | translocator-like | | |
| 00011 | Casein kinase 1, gamma 2 | 19p13.3 | NM_001319 |
| 00012 | Eukaryotic translation initiation | Xp22.12 | NM_001412 |
| | factor 1A, X-linked | | |
| 00014 | DnaJ (Hsp40) homolog, subfamily | 9p13-p12 | NM_001539 |
| | A, member 1 | | |
| 00017 | Casein kinase 1, alpha 1 | 5q32 | NM_001892 |
| 00018 | Casein kinase 1, epsilon | 22q13.1 | NM_001894 |
| 00021 | Karyopherin (importin) beta 1 | 17q21.32 | NM_002265 |
| 00022 | Microtubule-associated protein 4 | 3p21 | NM_002375 |
| 00024 | Myosin, heavy polypeptide 3, | 17p13.1 | NM_002470 |
| | skeletal muscle, embryonic | | |
| 00025 | Neuronal PAS domain protein 2 | 2q11.2 | NM_002518 |
| 00026 | Ornithine decarboxylase 1 | 2p25 | NM_002539 |
| 00027 | Period homolog 1 (Drosophila) | 17p13.1- | NM_002616 |
| | | 17p12 | |
| 00028 | Phosphomannomutase 1 | 22q13.2 | NM_002676 |
| 00029 | Proteasome (prosome, | 15q25.1 | NM_002789 |
| | macropain) subunit, alpha type, 4 | | |
| 00031 | Spectrin, alpha, erythrocytic 1 | 1q21 | NM_003126 |
| | (elliptocytosis 2) | | |
| 00032 | Signal transducer and activator of | 17q11.2 | NM_003152 |
| | transcription 5A | | |
| 00034 | SMT3 suppressor of mif two 3 | 2q33 | NM_003352 |
| | homolog 1 (yeast) | | |
| 00038 | Basic helix-loop-helix domain | 3p26 | NM_003670 |
| | containing, class B, 2 | | |
| 00039 | Timeless homolog (Drosophila) | 12q12-q13 | NM_003920 |
| 00040 | Cryptochrome 1 (photolyase-like) | 12q23-q24.1 | nm_004075 |
| 00043 | Succinate dehydrogenase | 5p15 | NM_004168 |
| | complex, subunit A, flavoprotein | | |
| | (Fp) | | |
| 00044 | Runt-related transcription factor 2 | 6p21 | NM_004348 |
| 00045 | Casein kinase 1, gamma 3 | 5q23 | NM_004384 |
| 00047 | Clock homolog (mouse) | 4q12 | NM_004898 |
| 00051 | V-ets erythroblastosis virus E26 | 21q22.3 | NM_005239 |
| | oncogene homolog 2 (avian) | | |
| 00053 | Protein phosphatase 1, regulatory | 10q23-q24 | NM_005398 |
| | (inhibitor) subunit 3C | | |
| 00054 | Nuclear receptor coactivator 4 | 10q11.2 | NM_005437 |
| 00057 | Melatonin receptor 1A | 4q35.1 | NM_005958 |
| 00060 | Tumor necrosis factor, alpha- | 14q32 | NM_006291 |
| | induced protein 2 | | |
| 00062 | UDP-glucose pyrophosphorylase | 2p14-p13 | NM_006759 |
| | 2 | | |
| 00063 | Quaking homolog, KH domain | 6q26-27 | NM_006775 |
| | RNA binding (mouse) | | |
| 00064 | Splicing factor 3a, subunit 3, | 1p34.3 | NM_006802 |
| | 60 kDa | | |
| 00066 | RNA binding protein with multiple | 8p12-p11 | NM_006867 |
| | splicing | | |
| 00067 | RAR-related orphan receptor B | 9q22 | NM_006914 |
| 00068 | SMT3 suppressor of mif two 3 | 21q22.3 | NM_006936 |
| | homolog 3 (yeast) | | |
| 00070 | Chromobox homolog 3 (HP1 | 7p15.2 | NM_007276 |
| | gamma homolog, Drosophila) | | |
| 00072 | Upstream binding transcription | 17q21.3 | NM_014233 |
| | factor, RNA polymerase I | | |
| 00079 | WD repeat domain 50 | 17q21.33 | NM_016001 |
| 00080 | Zinc finger RNA binding protein | 5p13.3 | NM_016107 |
| 00081 | Period homolog 3 (Drosophila) | 1p36.23 | NM_016831 |
| 00082 | Plexin A3 | Xq28 | NM_017514 |
| 00083 | 5-hydroxytryptamine (serotonin) | 10q21-q24 | NM_019859 |
| | receptor 7 (adenylate cyclase- | | |
| | coupled) | | |
| 00084 | Aryl hydrocarbon receptor nuclear | 12p12.2- | NM_020183 |
| | translocator-like 2 | p11.2 | |
| 00085 | Cryptochrome 2 (photolyase-like) | 11p11.2 | NM_021117 |
| 00086 | Nuclear receptor subfamily 1, | 17q11.2 | NM_021724 |
| | group D, member 1 | | |
| 00087 | Casein kinase 1, gamma 1 | 15q22.1- | NM_022048 |
| | | q22.31 | |
| 00088 | Period homolog 2 (Drosophila) | 2q37.3 | NM_022817 |
| 00090 | Basic helix-loop-helix domain | 12p11.23- | NM_030762 |
| | containing, class B, 3 | p12.1 | |
| 00092 | Zinc finger protein 384 | | NM_133476 |
| 00094 | Casein kinase 1, delta | 17q25 | NM_139062 |
| 00102 | SMT3 suppressor of mif two 3 | 17q25.1 | NM_006937 |
| | homolog 2 (yeast) | | |

### EXAMPLE 2

### Identification of Serum Markers for Alzheimer's Disease Involved in the Phenomenon of Phagocytosis

A DATAS analysis between the RNA extracted from patients suffering from Alzheimer's disease on the one hand and control patients on the other hand also demonstrated a change in the splicing of genes involved in the phenomenon of phagocytosis. This phenomenon, regulated by the signaling cascades that control oxidative stress, is representative of macrophages and their contribution to the phenomenon of innate immunity.

A decrease in the properties of phagocytosis of macrophages has been reported in certain patients suffering from Alzheimer's disease. However, no molecular basis has been identified to explain this phenomenon.

The DATAS analysis performed by the inventors made it possible to identify sequences corresponding to the RNA of L-plastin and calnexin. More particularly, the sequence identified for L-plastin corresponds to nucleotides 3249-3487 of GenBank sequence NM_{—}002298 and the sequence identified for calnexin corresponds to nucleotides 3764-4007 of GenBank sequence NM_{—}001746.

Calnexin is known by those persons skilled in the art to interact with presenilin 1 (PS1), a protein involved in Alzheimer's disease and in the production of amyloid-beta plaque. Calnexin also interacts with the CD14 receptor and is involved in the phenomenon of phagocytosis.

L-plastin, involved in the phenomenon of phagocytosis, is more particularly involved in the activation of oxidative stress consecutive to internalization.

Consequently, the present invention provides for the first time information on the molecular origin of defects in phagocytosis exhibited by the macrophages of patients suffering from Alzheimer's disease.

Table 2 below gives a list of the genes involved in the mechanisms of phagocytosis and oxidative stress, which constitute targets in the context of the present invention.

TABLE 2
L-Plastin - Genbank NM_002298
Calnexin - Genbank NM_001746
Homo sapiens CD14 molecule (CD14), transcript variant 1, mRNA.
ACCESSION NM_000591
Homo sapiens CD14 molecule (CD14), transcript variant 2, mRNA.
ACCESSION NM_001040021
Homo sapiens toll-like receptor 4 (TLR4), mRNA.
ACCESSION NM_138554
Homo sapiens toll-like receptor 2 (TLR2), mRNA.
ACCESSION NM_003264
Homo sapiens toll-like receptor 7 (TLR7), mRNA.
ACCESSION NM_016562
Homo sapiens chemokine (C-X-C motif) receptor 4 (CXCR4), transcript
variant 2, mRNA.
ACCESSION NM_003467
Homo sapiens chemokine (C-X-C motif) receptor 4 (CXCR4), transcript
variant 1, mRNA.
ACCESSION NM_001008540
Homo sapiens interferon, gamma (IFNG), mRNA.
ACCESSION NM_000619
Homo sapiens chemokine (C-X-C motif) ligand 3 (CXCL3), mRNA.
ACCESSION NM_002090
Homo sapiens interferon, beta 1, fibroblast (IFNB1), mRNA.
ACCESSION NM_002176
Homo sapiens interferon regulatory factor 3 (IRF3), mRNA.
ACCESSION NM_001571
Homo sapiens ras-related C3 botulinum toxin substrate 1 (rho
family, small GTP binding protein Rac1) (RAC1), transcript variant
Rac1c, mRNA.
ACCESSION NM_198829
Homo sapiens ras-related C3 botulinum toxin substrate 1 (rho
family, small GTP binding protein Rac1) (RAC1), transcript variant
Rac1, mRNA.
ACCESSION NM_006908
Homo sapiens ras-related C3 botulinum toxin substrate 1 (rho
family, small GTP binding protein Rac1) (RAC1), transcript variant
Rac1b, mRNA.
ACCESSION NM_018890
Homo sapiens lipopolysaccharide binding protein (LBP), mRNA.
ACCESSION NM_004139
Homo sapiens interleukin 1 receptor-like 1 (IL1RL1), transcript
variant 2, mRNA.
ACCESSION NM_003856
Homo sapiens interleukin 1 receptor-like 1 (IL1RL1), transcript
variant 1, mRNA.
ACCESSION NM_016232
Homo sapiens toll-like receptor 9 (TLR9), transcript variant B,
mRNA.
ACCESSION NM_138688
Homo sapiens toll-like receptor 9 (TLR9), transcript variant A,
mRNA.
ACCESSION NM_017442
Homo sapiens NADPH oxidase 4 (NOX4), mRNA.
ACCESSION NM_016931
Homo sapiens interleukin 6 (interferon, beta 2) (IL6), mRNA.
ACCESSION NM_000600
Homo sapiens macrophage migration inhibitory factor
(glycosylation-inhibiting factor) (MIF), mRNA.
ACCESSION NM_002415
Homo sapiens ras homolog gene family, member A (RHOA), mRNA.
ACCESSION NM_001664
Homo sapiens nuclear factor of kappa light polypeptide gene
enhancer in B-cells 1 (p105) (NFKB1), mRNA.
ACCESSION NM_003998
Homo sapiens interleukin 1, beta (IL1B), mRNA.
ACCESSION NM_000576
Homo sapiens interferon, alpha 1 (IFNA1), mRNA.
ACCESSION NM_024013
Homo sapiens nuclear factor of kappa light polypeptide gene
enhancer in B-cells inhibitor, alpha (NFKBIA), mRNA.
ACCESSION NM_020529
Homo sapiens interleukin 4 (IL4), transcript variant 2, mRNA.
ACCESSION NM_172348
Homo sapiens interleukin 4 (IL4), transcript variant 1, mRNA.
ACCESSION NM_000589
Homo sapiens PRotein Associated with Tlr4 (MGC40499), mRNA.
ACCESSION NM_152755
Homo sapiens toll-like receptor 3 (TLR3), mRNA.
ACCESSION NM_003265
Homo sapiens toll-like receptor 5 (TLR5), mRNA.
ACCESSION NM_003268
Homo sapiens interleukin 1 receptor accessory protein-like 1
(IL1RAPL1), mRNA.
ACCESSION NM_014271
Homo sapiens toll-like receptor 1 (TLR1), mRNA.
ACCESSION NM_003263
Homo sapiens interleukin 1 receptor-like 2 (IL1RL2), mRNA.
ACCESSION NM_003854
Homo sapiens chemokine (C-X-C motif) ligand 10 (CXCL10), mRNA.
ACCESSION NM_001565
Homo sapiens interleukin-1 receptor-associated kinase 1 (IRAK1),
transcript variant 3, mRNA.
ACCESSION NM_001025243
Homo sapiens interleukin-1 receptor-associated kinase 1 (IRAK1),
transcript variant 2, mRNA.
ACCESSION NM_001025242
Homo sapiens interleukin-1 receptor-associated kinase 1 (IRAK1),
transcript variant 1, mRNA.
ACCESSION NM_001569
Homo sapiens toll-like receptor adaptor molecule 2 (TICAM2), mRNA.
ACCESSION NM_021649
Homo sapiens toll interacting protein (TOLLIP), mRNA.
ACCESSION NM_019009
Homo sapiens toll-like receptor 8 (TLR8), transcript variant 2,
mRNA.
ACCESSION NM_138636
Homo sapiens toll-like receptor 8 (TLR8), transcript variant 1,
mRNA.
ACCESSION NM_016610
Homo sapiens interleukin 1 receptor accessory protein-like 2
(IL1RAPL2), mRNA.
ACCESSION NM_017416
Homo sapiens interleukin-1 receptor-associated kinase 4 (IRAK4),
mRNA.
ACCESSION NM_016123
Homo sapiens toll-like receptor 6 (TLR6), mRNA.
ACCESSION NM_006068
Homo sapiens toll-like receptor 10 (TLR10), transcript variant 2,
mRNA.
ACCESSION NM_001017388
Homo sapiens toll-like receptor 10 (TLR10), transcript variant 1,
mRNA.
ACCESSION NM_030956
Homo sapiens interleukin-1 receptor-associated kinase 2 (IRAK2),
mRNA.
ACCESSION NM_001570
Homo sapiens myeloid differentiation primary response gene (88)
(MYD88), mRNA.
ACCESSION NM_002468
Homo sapiens CD55 molecule, decay accelerating factor for
complement (Cromer blood group) (CD55), mRNA.
ACCESSION NM_000574

1. Method for detecting in vitro or ex vivo the presence of or the risk of developing Alzheimer's disease in a mammal, comprising the determination of the presence, in a biological sample from the mammal, preferably in a sample (derivative) of blood, of a change in one or more genes or RNA chosen among the genes regulated according to a circadian rhythm and the genes involved in regulating phagocytosis or oxidative stress, with the presence of such a change being indicative of the presence of or the risk of developing Alzheimer's disease in this mammal.

2. Method for evaluating or monitoring the effectiveness of a treatment for Alzheimer's disease, comprising a step of measuring the expression of one gene or, preferably, several genes chosen among the genes regulated according to a circadian rhythm and the genes involved in regulating phagocytosis or oxidative stress, during treatment, and comparing the expression thus measured with that measured at a previous stage of treatment.

3. Method according to claim 1, characterized in that the change in the gene or RNA is a change in the expression, splicing and/or structure of the protein produced.

4. Method according to claim 1, comprising the determination of the presence, in a biological sample from a mammal, preferably in a sample (derivative) of blood, of a change in one or more genes indicated in table 1, or in the corresponding RNA, in particular of a change in the splicing of one such gene or RNA.

5. Method according to claim 4, comprising the determination of the presence, in a biological sample from a mammal, preferably in a sample (derivative) of blood, of a change in the BMAL1 or CLOCK gene, or in a corresponding RNA, in particular of a change in the splicing of one such gene or RNA.

6. Method according to claim 1, comprising the determination of the presence, in a biological sample from a mammal, preferably in a sample (derivative) of blood, of a change in one or more genes indicated in table 2, or in the corresponding RNA, in particular of a change in the splicing of one such gene or RNA.

7. Method according to claim 6, comprising the determination of the presence, in a biological sample from a mammal, preferably in a sample (derivative) of blood, of a change in the L-plastin or calnexin gene, or in a corresponding RNA, in particular of a change in the splicing of one such gene or RNA.

8. Method according to claim 1, comprising the determination of the presence of one or more target molecules selected among:
a) the genes indicated in tables 1 and 2, or the corresponding RNAs, or a distinctive fragment of same having at least 15, preferably at least 16, 17, 18, 19, 20, 25 or 30 consecutive bases,
b) the nucleic acids having a sequence complementary to a sequence according to a),
c) the functional analogues of the nucleic acids according to a) or b), or
d) the polypeptides coded by the nucleic acids according to a) to c).

9. Method according to claim 1, characterized in that the presence of the change is determined by selective hybridization, selective amplification, or binding with a specific ligand.

10. Product comprising a substrate on which are immobilized nucleic acids comprising a sequence complementary to and/or specific of at least 5, 10, 20, 30, 40, 50, 60 or more genes or RNAs such as defined in claim 4.

11. Product comprising a substrate on which are immobilized at least 5, 10, 20, 30, 40, 50, 60 or more different polypeptide ligands coded by genes or RNAs such as defined in claim 4.

12. Kit comprising a compartment or container comprising at least 5, 10, 20, 30, 40, 50, 60 or more different nucleic acids and/or ligands chosen among the nucleic acids and ligands defined in claim 10.

13. Use of a product or kit according to claim 10 for detecting Alzheimer's disease in a mammalian subject, preferably a human subject.

14. Use of an acetylcholinesterase inhibitor, such as tacrine, donepezil or rivastigmine, for preparing a drug to treat Alzheimer's disease in a patient with dysregulation of the expression of a gene chosen among the genes regulated according to a circadian rhythm and the genes involved in regulating phagocytosis or oxidative stress.
